(19) United States
(12) Patent Application Publication
(10) Pub. No.: US 2018/0121784 A1
(43) Pub. Date: May 3, 2018

(54) CONVERSATION CONTROL SYSTEM

(71) Applicant: FUJI XEROX CO., LTD., TOKYO (JP)

(72) Inventors: Akira ICHIBOSHI, Kanagawa (JP); Roshan THAPLIYA, Kanagawa (JP)

(73) Assignee: FUJI XEROX CO., LTD., TOKYO (JP)

(21) Appl. No.: 15/647,279

(22) Filed: Jul. 12, 2017

(30) Foreign Application Priority Data
Oct. 27, 2016 (JP) 2016-210313

(51) Int. Cl. G06N 3/00; G09B 7/06; A61B 5/024; A61B 5/0295; G06N 5/04

(52) U.S. Cl. CPC G06N 3/006; G09B 7/06; A61B 2503/12; A61B 5/0295; G06N 5/04; A61B 5/024

(57) ABSTRACT

A conversation control system includes a conversation device, an acquisition unit that acquires personality information of a user that is registered in advance, a detection unit that detects biological information of the user, an estimation unit that estimates a mental state of the user from the acquired personality information and the detected biological information, and a changing unit that changes a personality of the conversation device in accordance with the estimated mental state of the user.

### CROSS-REFERENCE TO RELATED APPLICATIONS

This application is based on and claims priority under 35 USC 119 from Japanese Patent Application No. 2016-210313 filed Oct. 27, 2016.

### BACKGROUND

### Technical Field

The present invention relates to a conversation control system.

### SUMMARY

According to an aspect of the invention, there is provided a conversation control system including a conversation device, an acquisition unit that acquires personality information of a user that is registered in advance, a detection unit that detects biological information of the user, an estimation unit that estimates a mental state of the user from the acquired personality information and the detected biological information, and a changing unit that changes a personality of the conversation device in accordance with the estimated mental state of the user.

### BRIEF DESCRIPTION OF THE DRAWINGS

Exemplary embodiment(s) of the present invention will be described in detail based on the following figures, wherein:

FIG. 1 is a diagram illustrating an example of a conversation control system 10 according to an exemplary embodiment of the present invention;

FIG. 2 is a diagram illustrating a hardware configuration of a conversation type robot 20 in the exemplary embodiment;

FIG. 3 is a functional block diagram of the conversation type robot 20 in the exemplary embodiment;

FIG. 4 is a diagram illustrating a hardware configuration of a control server 40 in the exemplary embodiment;

FIG. 5 is a functional block diagram of the control server 40 in the exemplary embodiment;

FIG. 6 is a diagram illustrating an example of a user personality database 417 of the exemplary embodiment;

FIG. 7 is a flow chart illustrating an example of a flow of a conversation control process in the conversation control system 10 of this exemplary embodiment;

FIG. 8 is a conceptual diagram illustrating a mental state of a user 60 estimated on the basis of biological data acquired from a biological sensor 70;

FIG. 9 is a conceptual diagram illustrating a method of estimating a mental state at the present point in time by considering a mental state based on biological data obtained from the biological sensor 70 with respect to a personality of the user 60 at normal times, that is, a mental tendency; and

FIG. 10 is a flowchart illustrating another example of a flow of the conversation control process in the control server 40 of this exemplary embodiment.

### DETAILED DESCRIPTION

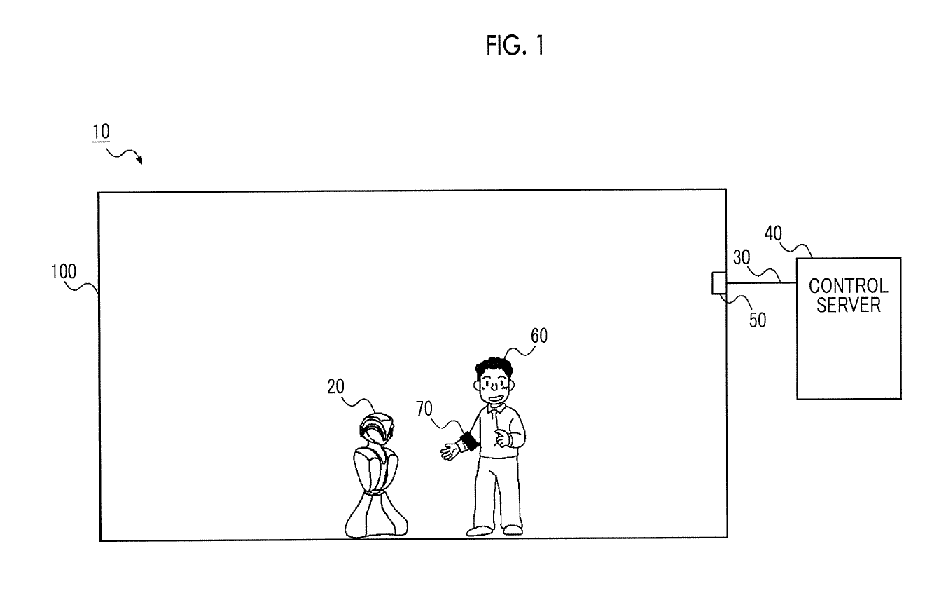

A conversation control system 10 according to an exemplary embodiment of the present invention will be described with reference to FIG. 1. The conversation control system 10 of this exemplary embodiment is configured to include a conversation type robot 20 disposed in a comparatively large predetermined area (hereinafter, referred to as a work place) 100 such as the floor of an office building, and a control server 40. The control server 40 is connected to the conversation type robot 20 through a network 30 and an access point 50 installed on a wall surface of the workplace 100 in a wireless manner. Further, a user 60 is present in the work place 100, a biological sensor 70 is attached to a wrist or an arm of the user 60, and the biological sensor 70 and the control server 40 are connected to each other through the access point 50 in a wireless manner.

The biological sensor 70 detects a physical symptom of the current emotion of the user 60, for example, biological information. The biological information includes, for example, at least one of the skin potential, the heart rate, and data regarding volume pulse waves of peripheral blood vessels of the user 60. Information regarding the skin potential includes the displacement and distribution of the skin potential at normal times and a variation in the skin potential per unit time in addition to the value of the current skin potential. Similarly, information regarding the heart rate includes the displacement of the heart rate at normal times and a variation in the heart rate per unit time in addition to the current heart rate. In addition, the data regarding the volume pulse waves of the peripheral blood vessels includes data regarding the contraction and expansion of the current blood vessel.

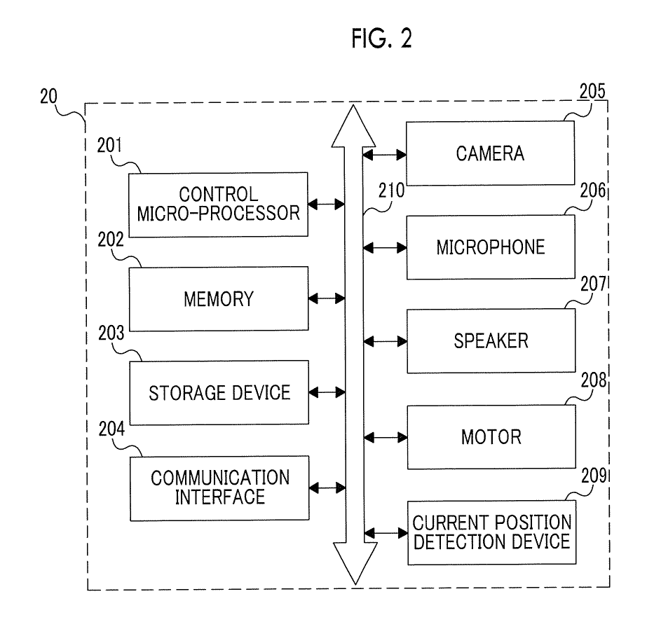

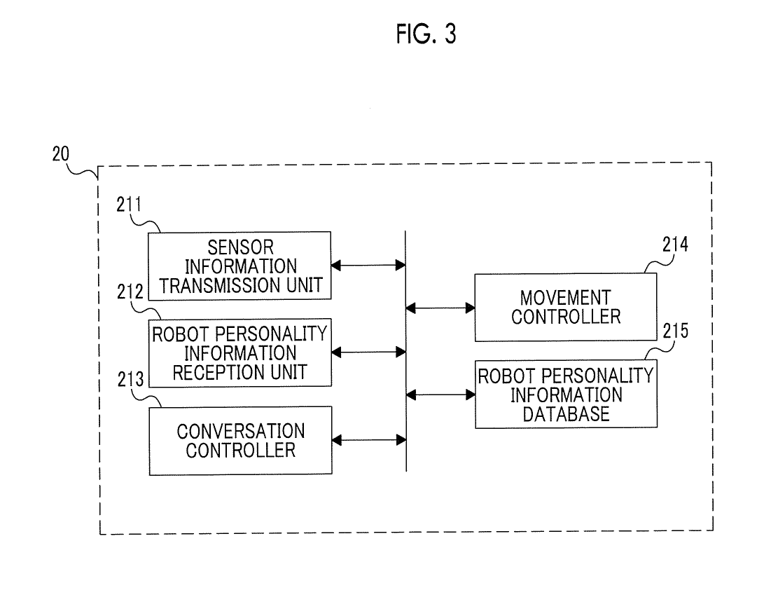

First, the conversation type robot 20 of this exemplary embodiment will be described with reference to FIGS. 2 and 3. FIG. 2 is a diagram illustrating a hardware configuration of the conversation type robot 20. As illustrated in FIG. 2, the conversation type robot 20 is configured to include a control micro-processor 201, a memory 202, a storage device 203 such as a hard disk drive (HDD) or a solid state drive (SSD), a communication interface 204, a camera 205, a microphone 206, a speaker 207, a motor 208, and a current position detection device 209, which are connected to a control bus 210.

The control micro-processor 201 controls the overall operation of the components of the conversation type robot 20 on the basis of a control program stored in the storage device 203. The memory 202 temporarily stores conversation sounds during a conversation between the conversation type robot 20 and the user, conversation contents, a photo of the face, images of a facial expression, a behavior, and the physical state of the user 60 which are captured by the camera 205, and the like. The storage device 203 stores a control program for controlling each unit of the conversation type robot 20. The communication interface 204 performs communication control for causing the conversation type robot 20 to communicate with the control server 40 through the access point 50.

The camera 205 captures the facial image, the facial expression, the behavior, a change in the physical state of the user, and the like, and stores the captured images in the memory 202. The microphone 206 detects the user's sound during a conversation with the user and stores, that is, records the detected sound in the memory 202. The memory 202 may store conversation contents after the analysis of sound contents and the pitch of a sound or the speed of words, instead of directly recording a sound. The speaker 207 outputs a sound generated by a conversation controller, to be described later, of the conversation type robot 20. The motor 208 moves the conversation type robot 20 to a predetermined position on the basis of movement control information generated in a movement controller to be described later. The current position detection device 209, which is configured to include an acceleration sensor, a GPS signal reception device, a positional information signal reception device, and the like, specifies the current position of the conversation type robot 20 and temporarily stores the specified current position in the memory 202.

FIG. 3 is a functional block diagram of the conversation type robot 20. The conversation type robot 20 executes a control program stored in the storage device 203 in the control micro-processor 201 to function as a sensor information transmission unit 211, a robot personality information reception unit 212, a conversation controller 213, a movement controller 214, and a robot personality information database 215 as illustrated in FIG. 3.

The sensor information transmission unit 211 transmits the photo of the face of the user 60 which is captured by the camera 205 of the conversation type robot 20 and external information of the user 60 which is detected by the camera 205 and the microphone 206 to the control server 40. The external information includes data regarding a facial expression and a behavior of the user 60 which are captured by the camera 205, and data regarding the pitch of a sound and the speed of words of the user 60 which are detected by the microphone 206. Meanwhile, a portion of the external information, for example, the angles of the mouth and eyebrows of the user 60, the number of blinks, information regarding a body temperature obtained by analyzing an RGB image of the user 60 which is captured by a camera, and information such as the pitch of a sound can also be handled as biological information, but any of the external information and the biological information is transmitted to the control server 40 by the sensor information transmission unit 211.

The robot personality information reception unit 212 receives information regarding a personality to be taken by the conversation type robot 20, which is transmitted from a robot personality information transmission unit of the control server 40 to be described later, and temporarily stores the received information in the memory 202.

The conversation controller 213 controls conversation performed between the conversation type robot 20 and the user 60. Specifically, the conversation controller 213 generates a response message in accordance with a conversation method and conversation contents based on the personality to be taken by the robot which is received by the robot personality information reception unit 212 with reference to the robot personality information database 215 to be described later and outputs the generated response message to the speaker 207, or controls the driving of the motor 208 and changes the posture or behavior of the conversation type robot 20.

The movement controller 214 controls the movement of the conversation type robot 20. The movement controller 214 generates movement control information regarding movement from the current position to a target location in a case where an instruction for movement is given from the control server 40, controls the operation of the motor 208 while referring to information regarding the current position detected by the current position detection device 209, and moves the conversation type robot 20.

The robot personality information database 215 stores a conversation method and response contents of the conversation type robot 20 for each personality to be taken by the conversation type robot 20.

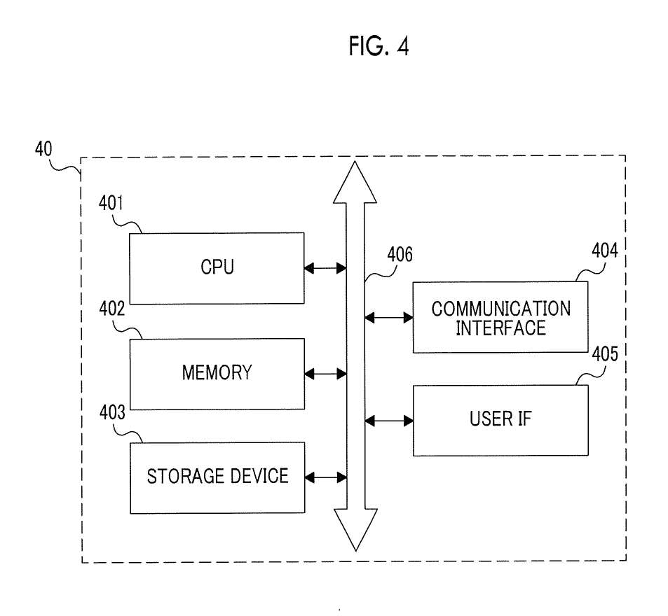

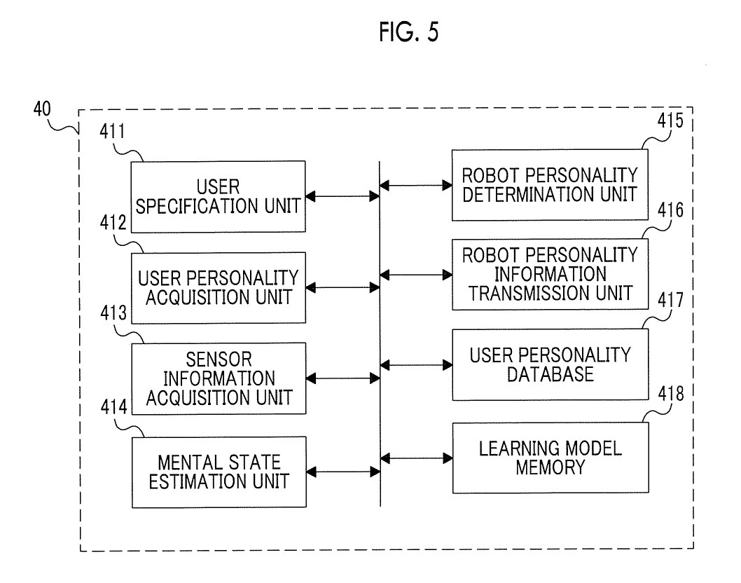

Next, the control server 40 of this exemplary embodiment will be described with reference to FIGS. 4 and 5. FIG. 4 is a diagram illustrating a hardware configuration of the control server 40. As illustrated in FIG. 4, the control server 40 is configured to include a CPU 401, a memory 402, a storage device 403, a communication interface 404, and a user interface 405, which are connected to a control bus 406.

The CPU 401 controls the overall operation of the components of the control server 40 on the basis of a control program stored in the storage device 403. The memory 402 stores positional information of the conversation type robot 20 which is transmitted from the conversation type robot 20, a photo of the face, external information, or biological information of the user 60, and biological information of the user 60 which is transmitted from the biological sensor 70 attached to the user 60.

The storage device 403 is a hard disk drive (HDD), a solid state drive (SSD), or the like, and stores a control program for controlling the control server 40. Further, although will be described later, the storage device 403 stores a machine learning model which is used when a user personality database or the control server 40 estimates the current mental state of the user 60.

The communication interface 404 performs communication control for the control server 40 to transmit and receive various data to and from the conversation type robot 20 and the biological sensor 70 attached to the user 60 through the access point 50. The user interface 405 is constituted by a display device such as a liquid crystal display and an input device such as a keyboard or a mouse, and is used to make a manager control the control program stored in the storage device 403.

FIG. 5 illustrates a functional block diagram of the control server 40. The control server 40 executes the control program stored in the storage device 403 in the CPU 401 to function as a user specification unit 411, a user personality acquisition unit 412, a sensor information acquisition unit 413, a mental state estimation unit 414, a robot personality determination unit 415, a robot personality information transmission unit 416, a user personality database 417, and a learning model memory 418 as illustrated in FIG. 5.

The user specification unit 411 specifies who the user 60 is as a conversation party of the conversation type robot 20 on the basis of the photo of the face of the user 60 which is transmitted from the sensor information transmission unit 211 of the conversation type robot 20. Meanwhile, the specification of the user 60 may adopt a method using voiceprint authentication for analyzing sound data in addition to a method using the photo of the face.

The user personality acquisition unit 412 acquires personality information at normal times representing a mental tendency of the user 60 at normal times which is specified by the user specification unit 411 from the user personality database 415. These pieces of personality information of the respective users at normal times may be stored in the user personality database 417 by causing the user personality acquisition unit 412 to analyze results of a personality diagnosis test or a questionnaire performed on each of the users in advance. Alternatively, the user personality acquisition unit 412 may perform a personality diagnosis test on the user 60 in advance through the conversation type robot 20 and analyze the result thereof to generate personality information of the user 60 at normal times and store the generated personality information in the user personality database 417.

The sensor information acquisition unit 413 receives external information and biological information of the user which are transmitted from the sensor information transmission unit 211 of the conversation type robot 20 and biological information transmitted from the biological sensor 70, and stores the received information in the memory 402.

The mental state estimation unit 414 inputs the personality information of the user 60 at normal times which is acquired by the user personality acquisition unit 412 and the external information and the biological information of the user 60 which are acquired by the sensor information acquisition unit 413 to a machine learning model stored in the learning model memory 418 to be described later, and obtains the mental state of the user 60 as an output, thereby estimating the current mental state.

The robot personality determination unit 415 determines a personality to be taken by the conversation type robot 20 in accordance with the current mental state of the user 60 which is estimated by the mental state estimation unit 414. A correspondence table (not shown) storing various current mental states of the user 60 and personalities to be taken by the conversation type robot 20 in association with each other is generated in advance, and the robot personality determination unit 415 determines a personality to be taken by the conversation type robot 20 with reference to the correspondence table. For example, when the current mental state of the user 60 is “introvert and stable”, a personality to be taken by the conversation type robot 20 is set to be “introvert and stable”. This correspondence table may be manually created by a manager, or may be generated by machine learning. In a case where the correspondence table is generated by machine learning, the users 60 having various personalities (mental states) are caused to have a conversation with the conversation type robot 20 exhibiting various personalities, biological information detected by the biological sensor 70 attached to the user 60 or the camera 205 of the conversation type robot 20 is analyzed, and a personality of the conversation type robot 20 which is estimated to make the user 60 of each of the personalities feel comfortable is registered in the correspondence table.

The robot personality information transmission unit 416 transmits the personality to be taken by the conversation type robot 20 which is determined by the robot personality determination unit 415 to the conversation type robot 20.

The user personality database 417 stores personality information at normal times representing a mental tendency at normal times for each user. For example, the personality information of the user at normal times is represented by a diplomatic scale, a neurotic scale, and a psychotic scale, and is stored as a numerical value for each user. Meanwhile, the personality information of the user at normal times is not limited to the scales represented by the above-described elements, and may be represented by another scale such as a mental stability scale, a social adaptation scale, or an impulsive scale.

The learning model memory 418 stores a machine learning model. The machine learning model outputs the current mental state of the user in a case where personality information of the user at normal times indicating a mental tendency at normal times and the current biological information of the user are input.

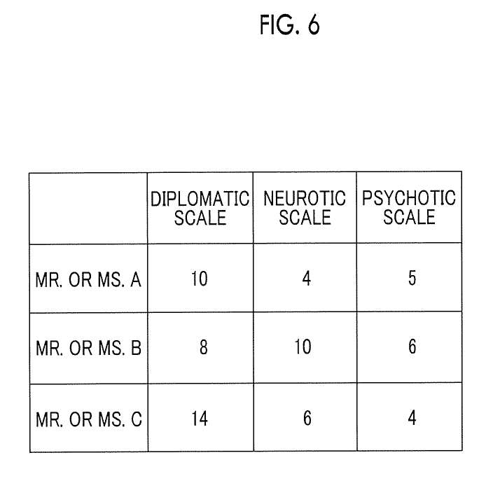

FIG. 6 is a diagram illustrating an example of the user personality database 417. Referring to FIG. 6, a “diplomatic scale”, a “neurotic scale”, and a “psychotic scale” of each of “Mr. or Ms. A” to “Mr. or Ms. C” are digitized and registered. These numerical values are registered by performing a questionnaire on the user 60 having a possibility of having a conversation with the conversation type robot 20 and causing a manager to manually input the result thereof to the control server 40. Alternatively, the user personality acquisition unit 412 of the control server 40 may instruct the conversation type robot 20 to perform a personality diagnosis test, and personality information at normal times representing a mental tendency of the user 60 at normal times based on the results may be digitized. In this case, the conversation controller 213 of the conversation type robot 20 performs the personality diagnosis test while having a conversation with a user 60 on the basis of the instruction for performing the personality diagnosis test which is received from the control server 40, and transmits a reply of the user 60 to the control server 40. The user personality acquisition unit 412 of the control server 40 digitizes personality information of each of the users 60 at normal times on the basis of the reply of the personality diagnosis test, and registers the digitized personality information in the user personality database 417.

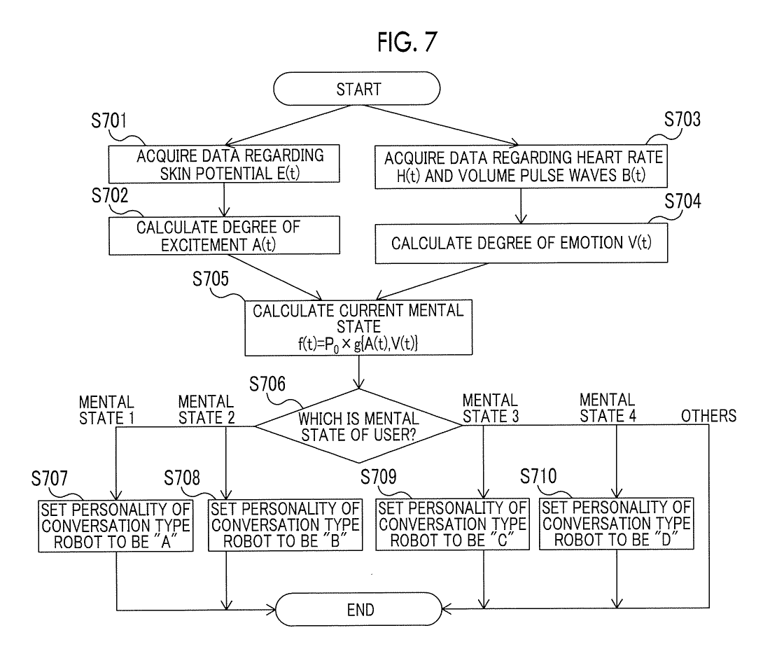

Next, a flow of a conversation control process in the conversation control system 10 will be described with reference to FIG. 7. FIG. 7 is a flow chart illustrating an example of a flow of a conversation control process in the control server 40 of this exemplary embodiment. Meanwhile, it is assumed that a process of specifying the user 60 having a conversation with the conversation type robot 20 has been already performed by the user specification unit 411. In step S701, the sensor information acquisition unit 413 of the control server 40 acquires data E(t) regarding the skin potential of the user 60 from the biological sensor 70, and stores the acquired data in the memory 402. In the next step S702, the mental state estimation unit 414 calculates a degree of excitement A(t) at the present point in time on the basis of the data E(t) regarding the skin potential, and proceeds to step S705.

In step S703 performed in parallel with step S701, the sensor information acquisition unit 413 of the control server 40 acquires data H(t) regarding the heart rate and data B(t) regarding volume pulse waves of peripheral blood vessels of the user 60 from the biological sensor 70, and stores the acquired data in the memory 402. In the next step S704, the mental state estimation unit 414 calculates a degree of emotion V(t) at the present point in time on the basis of the data H(t) regarding the heart rate and the data B(t) of the volume pulse waves of the peripheral blood vessels, and proceeds to step S705.

In step S705, the mental state estimation unit 414 of the control server 40 estimates a mental state at the present point in time of the user 60. Specifically, the user personality acquisition unit 412 acquires personality information at normal times representing a mental tendency of the user 60 at normal times with reference to the user personality database 417. Further, the mental state estimation unit 414 calculates the degree of displacement of the mental state of the user 60 at the present point in time from personality information at normal times P₀ on the basis of the degree of excitement A (t) and the degree of emotion V(t) which are respectively calculated in steps S702 and S704. More specifically, a mental state f(t) is calculated by the following expression.

f(t)=P₀×g(A(t),V(t))

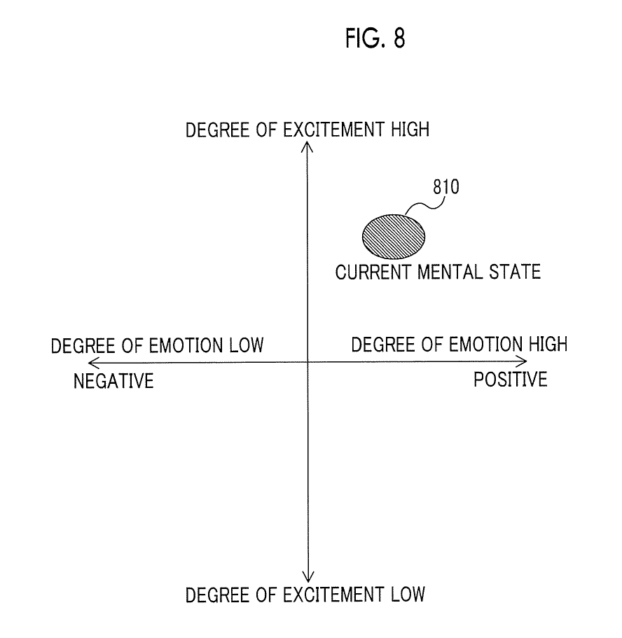

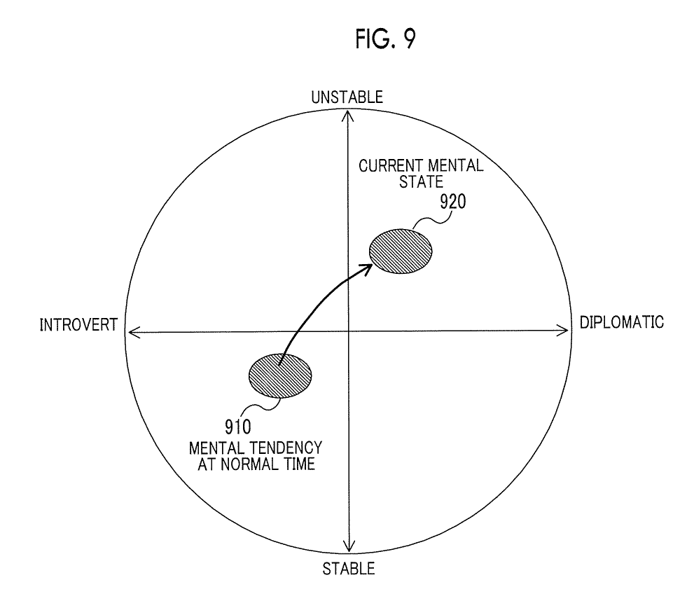

FIG. 8 is a conceptual diagram illustrating the mental state of the user 60 which is estimated on the basis of biological data acquired from the biological sensor 70. FIG. 9 is a conceptual diagram illustrating a method of estimating the mental state at the present point in time in consideration of a mental state based on the biological data acquired from the biological sensor 70 with respect to personality information of the user 60 at normal times. In FIG. 8, a degree of emotion V(t) is taken for the horizontal axis, and a degree of excitement A(t) is taken for the vertical axis. Therefore, the degree of emotion V(t) and the degree of excitement A(t) respectively calculated in steps S702 and S704 are plotted on FIG. 8, and thus it is possible to estimate a mental state 810 at the present point in time to a certain extent. However, in this exemplary embodiment, a mental state at the present point in time is estimated on the basis of the biological data acquired from the biological sensor 70 and data regarding the personality information of the user 60 at normal times.

In FIG. 9, the horizontal axis is represented by an introvert-diplomatic scale, and the vertical axis is represented by a stability-unstability scale. For example, the diplomatic scale of the user 60 which is acquired from the user personality database 417 of FIG. 6 is plotted in the horizontal axis as it is, and the neurotic scale is plotted in the vertical axis (stability-unstability scale), whereby it is possible to estimate a personality (mental tendency at normal times) 910 (P₀) at normal times. Further, a region displaced from the personality at normal times in the directions of the horizontal axis and the vertical axis by the degree of emotion V(t) and the degree of excitement A(t) which are calculated on the basis of the data acquired from the biological sensor 70 is estimated to be a current mental state 920. Meanwhile, the degree of emotion of FIG. 8 and the introvert-diplomatic scale of FIG. 9 are not necessarily associated with each other on a one-to-one basis. Similarly, the degree of excitement of FIG. 8 and the stability-unstability scale of FIG. 9 are also not necessarily associated with each other on a one-to-one basis. However, for convenience of description, a description will be given here on the assumption that the scales are substantially the same scales.

In step S706 of FIG. 7, the robot personality determination unit 415 of the control server 40 determines whether or not a mental state at the present point in time of the user 60 which is estimated in step S705 corresponds to any mental state determined in advance. In a case where it is determined that the mental state of the user 60 is a first mental state (for example, introvert and stable), the process proceeds to the processing of step S707, and the robot personality determination unit 415 determines a personality to be taken by the conversation type robot 20 to be a personality A (for example, introvert and stable, similar to the user 60) to generate robot personality information with reference to the above-described correspondence table. The generated robot personality information is transmitted to the conversation type robot 20 by the robot personality information transmission unit 416, and the process is terminated. Meanwhile, the robot personality determination unit 415 determines a personality to be taken by the conversation type robot 20 with reference to the above-described correspondence table. Further, the robot personality information reception unit 212 of the conversation type robot 20 receives the robot personality information transmitted from the control server 40, and the conversation controller 213 has a conversation with the user 60 by the determined personality of the robot while referring to the robot personality information database 215 on the basis of the received robot personality information.

In a case where it is determined in step S706 that the mental state of the user 60 is each of second to fourth mental states, the process proceeds to the processing of steps S708 to S710 in accordance with the determined mental states, and the robot personality determination unit 415 determines a personality to be taken by the conversation type robot 20 to be B to D and generates pieces of robot personality information corresponding to the respective personalities with reference to the above-described correspondence table. The robot personality information transmission unit 416 transmits the robot personality information to the conversation type robot 20, and the process is terminated.

In a case where it is determined in step S706 that the mental state of the user 60 does not correspond to any mental state determined in advance, the process is terminated.

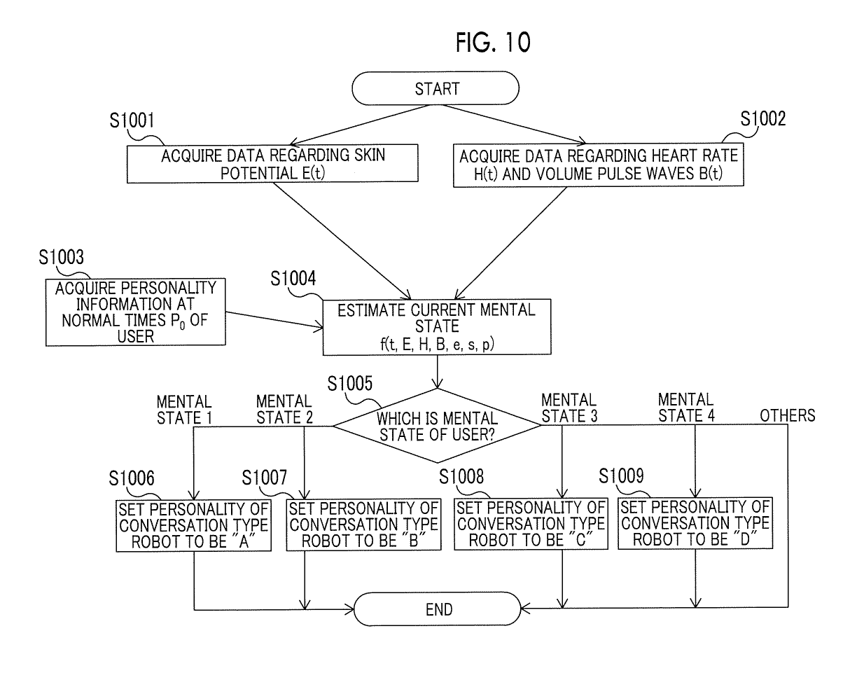

Next, another method of the conversation control process in the conversation control system 10 will be described with reference to FIG. 10. FIG. 10 is a flow chart illustrating another example of a flow of the conversation control process in the control server 40 of this exemplary embodiment. Meanwhile, it is assumed that a process of specifying the user 60 having a conversation with the conversation type robot 20 has been already performed by the user specification unit 411. In step S1001, the sensor information acquisition unit 413 of the control server 40 acquires data E(t) regarding the skin potential of the user 60 from the biological sensor 70, and stores the acquired data in the memory 402. Then, the process proceeds to step S1004. In step S1002 performed in parallel with step S1001, the sensor information acquisition unit 413 of the control server 40 acquires data H(t) regarding the heart rate and data B(t) regarding volume pulse waves of peripheral blood vessels of the user 60 from the biological sensor 70, and stores the acquired data in the memory 402. Then, the process proceeds to step S1004.

In step S1003 performed in parallel with steps S1001 and S1002, the user personality acquisition unit 412 acquires personality information at normal times P₀ (diplomatic scale e, neurotic scale s, and psychotic scale p) which represents a mental tendency of the user 60 at normal times with reference to the user personality database 417, and the process proceeds to step S1004.

In step S1004, the mental state estimation unit 414 inputs the data E(t) regarding the skin potential, the data H(t) regarding the heart rate, the data B(t) regarding the volume pulse waves of the peripheral blood vessels, and the personality information at normal times (e, s, p) of the user 60 which are acquired in steps S1001 to S1003 to the machine learning model stored in the learning model memory 418, and obtains a current mental state f(t) of the user 60 as an output, thereby estimating the current mental state.

In step S1005, the robot personality determination unit 415 of the control server 40 in step S1005 determines whether or not the estimated mental state at the present point in time of the user 60 corresponds to any mental state determined in advance. In a case where the mental state of the user 60 is a first mental state (for example, introvert and stable), the process proceeds to the processing of step S1006. The robot personality determination unit 415 determines a personality to be taken by the conversation type robot 20 to be a personality A (for example, introvert and stable, similar to the user 60) to generate robot personality information with reference to the above-described correspondence table. The generated robot personality information is transmitted to the conversation type robot 20 by the robot personality information transmission unit 416, and the process is terminated. Meanwhile, the robot personality information reception unit 212 of the conversation type robot 20 receives the robot personality information transmitted from the control server 40, and the conversation controller 213 has a conversation with the user 60 by the determined personality of the robot while referring to the robot personality information database 215 on the basis of the received robot personality information.

In a case where it is determined in step S1005 that the mental state of the user 60 is each of second to fourth mental states, the process proceeds to the processing of steps S1007 to S1009 in accordance with the determined mental states, and the robot personality determination unit 415 determines a personality to be taken by the conversation type robot 20 to be B to D and generates pieces of robot personality information corresponding to the respective personalities with reference to the above-described correspondence table. The robot personality information transmission unit 416 transmits the robot personality information to the conversation type robot 20, and the process is terminated.

In a case where it is determined in step S706 that the mental state of the user 60 does not correspond to any mental state determined in advance, the process is terminated.

Meanwhile, in the description of FIG. 7, a case where the degree of excitement A(t) and the degree of emotion V(t) of the user 60 are calculated on the basis of data measured by the biological sensor 70 has been described. In addition, in the description of FIG. 10, a case where the data acquired from the biological sensor 70 is input to the machine learning model has been described. However, the present invention is not limited to the above-described example, and external information or biological information of the user 60 may be detected by another sensor. For example, a facial expression, the number of blinks, and a body temperature of the user 60 may be detected by the camera 205 of the conversation type robot 20, the pitch of a sound of the user 60 may be detected by the microphone 206, the degree of excitement A(t) of the user 60 may be calculated on the basis of these detected data, and the degree of emotion V(t) of the user 60 may be calculated on the basis of the facial expression, body movement, and posture of the user 60 which are detected by the camera 205 and the pitch of a sound of the user 60 which is detected by the microphone 206.

In addition, in the description of FIG. 10, a case where the current mental state of the user 60 is estimated by inputting personality information at normal times representing a mental tendency of the user 60 at normal times and a physical symptom of the current emotion of the user 60 which is acquired from the biological sensor 70 to the machine learning model has been described. However, the present invention is not limited to the above-described example, and an output of another sensor may be input to the machine learning model. For example, the current mental state of the user is obtained as an output by inputting data regarding a facial expression (the angles of the mouth and eyebrows), the number of blinks, body temperature, body movement, and posture of the user 60 which are detected by the camera 205 of the conversation type robot 20 and data regarding the pitch of a sound of the user 60 which is detected by the microphone 206 to a machine learning model as biological information, and thus the current mental state of the user 60 may be estimated.

Meanwhile, in the above-described exemplary embodiment, a case where the conversation type robot 20 is used as a conversation device has been described. However, in the present invention, the conversation device may not only be the conversation type robot 20 but also a device having a conversation function, and may be, for example, a portable terminal device having a conversation function.

The foregoing description of the exemplary embodiments of the present invention has been provided for the purposes of illustration and description. It is not intended to be exhaustive or limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

1. A conversation system comprising:
an acquisition unit that acquires personality information of a user that is registered in advance;
a detection unit that detects biological information of the user;
an estimation unit that estimates a mental state of the user from the acquired personality information and the detected biological information; and
a changing unit that changes a conversation method and response contents of a conversation device in accordance with the estimated mental state of the user.

2. The conversation system according to claim 1,
wherein the estimation unit estimates the mental state of the user on the basis of a displacement from the personality information of the user that is obtained from the detected biological information of the user.

3. The conversation system according to claim 1,
wherein the conversation control system includes a machine learning model that inputs the biological information of the user and the personality information of the user and outputs the mental state of the user, and
wherein the estimation unit inputs current biological information and personality information of the user to the machine learning model to estimate a current mental state of the user as an output.

4. The conversation system according to claim 1,
wherein the detection unit includes a biological sensor and detects at least any one of a skin potential, a heart rate, and volume pulse waves of peripheral blood vessels of the user.

5. The conversation system according to claim 2,
wherein the detection unit includes a biological sensor and detects at least any one of a skin potential, a heart rate, and volume pulse waves of peripheral blood vessels of the user.

6. The conversation system according to claim 3,
wherein the detection unit includes a biological sensor and detects at least any one of a skin potential, a heart rate, and volume pulse waves of peripheral blood vessels of the user.

7. The conversation system according to claim 1,
wherein the detection unit includes a camera and detects at least any one of a facial expression, positional information of a part of a face, the number of blinks, a body temperature, a body action and a posture of the user.

8. The conversation system according to claim 2,
wherein the detection unit includes a camera and detects at least any one of a facial expression, positional information of a part of a face, the number of blinks, a body temperature, a body action, and a posture of the user.

9. The conversation system according to claim 3,
wherein the detection unit includes a camera and detects at least any one of a facial expression, positional information of a part of a face, the number of blinks, a body temperature, a body action, and a posture of the user.

10. The conversation system according to claim 4,
wherein the detection unit includes a camera and detects at least any one of a facial expression, positional information of a part of a face, the number of blinks, a body temperature, a body action, and a posture of the user.

11. The conversation system according to claim 5,
wherein the detection unit includes a camera and detects at least any one of a facial expression, positional information of a part of a face, the number of blinks, a body temperature, a body action, and a posture of the user.

12. The conversation system according to claim 1,
wherein the acquisition unit acquires the personality information of the user from results of a questionnaire or a personality diagnosis test.

13. The conversation system according to claim 2,
wherein the acquisition unit acquires the personality information of the user from results of a questionnaire or a personality diagnosis test.

14. The conversation system according to claim 3,
wherein the acquisition unit acquires the personality information of the user from results of a questionnaire or a personality diagnosis test.

15. The conversation system according to claim 4,
wherein the acquisition unit acquires the personality information of the user from results of a questionnaire or a personality diagnosis test.

16. The conversation system according to claim 5,
wherein the acquisition unit acquires the personality information of the user from results of a questionnaire or a personality diagnosis test.

17. The conversation system according to claim 6,
wherein the acquisition unit acquires the personality information of the user from results of a questionnaire or a personality diagnosis test.

18. The conversation system according to claim 7,
wherein the acquisition unit acquires the personality information of the user from results of a questionnaire or a personality diagnosis test.

19. The conversation system according to claim 1,
wherein the acquisition unit acquires a diplomatic-introvert scale, a neurotic scale, and a psychotic tendency scale as the personality information of the user.

20. The conversation system according to claim 1, further comprising:
a controller that performs control for causing the conversation device to have a conversation with the user in accordance with the changed conversation method and response contents.

21. The conversation system according to claim 1,
wherein the conversation method comprises a posture or a behavior of the conversation device.
